(19) United States
(12) Patent Application Publication
(10) Pub. No.: US 2011/0240031 A1
(43) Pub. Date: Oct. 6, 2011

(54) RESPIRATORY INTERFACE DEVICE INCLUDING A MECHANISM FOR MANAGING NOSE BRIDGE PRESSURE

(75) Inventors: Marcel Douglas Jaffre, Greensburg, PA (US); Anthony Startare, Belle Vernon, PA (US)

(73) Assignee: KONINKLIJKE PHILIPS ELECTRONICS, N.V., EINDHOVEN (NL)

(21) Appl. No.: 13/132,943

(22) PCT Filed: Nov. 21, 2009
(86) PCT No.: PCT/IB2009/055261

§ 371 Date: Jun. 6, 2011

Related U.S. Application Data
(60) Provisional application No. 61/139,725, filed on Dec. 22, 2008.

(51) Int. Cl. A61M 16/06

(52) U.S. Cl. 128/206.21

(57) ABSTRACT

A respiratory interface device includes a mask, a connector tubing component, a headgear and a tube management device coupled to the headgear. The connector tubing component has a main tubing portion and a swivel mechanism provided at an end thereof for coupling the connector tubing component to a gas supply tube. The main tubing portion of the connector tubing component is structured to be moveably received within and held by the tube management device. The pressure applied to the bridge of the nose of the user by the mask may be selectively adjusted by adjusting the position of the main tubing portion within the tube management device. The tube management device may, for example, be threaded, employ a friction fit, employ a cam mechanism, or employ a compressible tube attachment piece, among other implementations.

### CROSS-REFERENCE TO RELATED APPLICATIONS

This patent application claims the priority benefit under 35 U.S.C. §119(e) of U.S. Provisional Application No. 61/139,725 filed on Dec. 22, 2008, the contents of which are herein incorporated by reference.

### BACKGROUND OF THE INVENTION

1. Field of the Invention

The present invention relates to respiratory interface devices for transporting a gas to and/or from an airway of a user, and in particular, to a respiratory interface device including a mechanism for managing nose bridge pressure and mask removal.

2. Description of the Related Art

A variety of respiratory interface devices are known in the art. These interface devices include a mask through which gases can be provided (e.g., at a positive pressure) for consumption by the user. Such masks include, without limitation, nasal/oral masks that fit over the mouth and nose of the user, nasal masks which fit over only the nose of the user, and nasal pillows with prongs which fit into the nares of the user. It is known to maintain such interfaces on the face of a user by a headgear that wraps around the head of the user. The uses for such interface devices include high altitude breathing (aviation applications), swimming, mining, fire fighting and various medical diagnostic and therapeutic applications.

One requisite of many of these interface devices, (e.g.) particularly medical respiratory interface devices, is that they provide an effective fit against the user's face to limit or prevent leakage of the gas being supplied. Commonly, in conventional respiratory interface device configurations, an effective device-to-face fit has been attained in many instances only with considerable discomfort for the user. This problem is most crucial in those applications, especially medical applications, which require the user to wear the mask continuously for hours or perhaps even days. In such situations, the user often will not tolerate the mask for long durations and therefore optimum therapeutic or diagnostic objectives will not be achieved, or will be achieved with great difficulty and considerable user discomfort. One area where fit and comfort is often a concern is the bridge of the user's nose, as most interface devices will apply a pressure to this area. If this pressure is not able to be managed effectively, either or both of a poor fit or patient discomfort will result, thereby limiting the effectiveness of the device.

In addition, as is known, users of such interface devices, (e.g.) particularly medical respiratory interface devices, often have to fully or partially remove the device many times during use to, for example, talk, eat, take medication, or clean the device. Most current respiratory interface devices do not provide an efficient and convenient mechanism for fully or partially removing and re-applying the respiratory interface device.

### SUMMARY OF THE INVENTION

A respiratory interface device is provided that includes a mask structured to engage the bridge of the user's nose, a connector tubing component, a headgear and a tube management device coupled to the headgear. The first end of the connector tubing component is coupled to the mask. The connector tubing component has a main tubing portion and a swivel mechanism provided at the second end of the connector tubing component for coupling the connector tubing component to a gas supply tube in a manner that permits the connector tubing component and the gas supply tube to rotate relative to one another. The main tubing portion of the connector tubing component is structured to be moveably received within and held by the tube management device. The pressure applied to the bridge of the nose of the user by the mask may be selectively adjusted by adjusting the position of the main tubing portion within the tube management device. A second swivel mechanism may be provided at the first end of the connector tubing component for coupling the connector tubing component to the mask in a manner that permits the connector tubing component and the mask to rotate relative to one another.

In one particular embodiment, the tube management device includes a recess having a plurality of threads for receiving the main tubing portion, wherein the main tubing portion has an edge, such as a helical edge, extending along at least a portion of a length thereof for cooperating with the plurality of threads. The main tubing portion is structured to move within the tube management device in response to rotation of the second swivel mechanism as a result of the cooperation between the edge and the plurality of threads. In another particular embodiment, the main tubing portion is held within the tube management device by a friction force, wherein as a result of the friction force the main tubing portion is structured to move within the tube management device in a direction substantially along a longitudinal axis of the main tubing portion only in response to a pulling force of at least 3 lbs, or at least 4 lbs. In an alternative embodiment, the tube management device comprises a cam mechanism that employs a cam connected to a lever to selectively impart a clamping force onto the main tubing portion. In yet another alternative embodiment, the device further includes a selectively compressible tube attachment piece selectively attachable to the main tubing portion at a desired position along a length thereof, wherein the main tubing portion is coupled to the tube management device through the selectively compressible tube attachment piece.

In an exemplary embodiment, the connector tubing component is removable from the tube management device in response to a pulling force being applied to the connector tubing component in a direction that is substantially normal to the tube management device to provide a quick release feature that allows the mask to be rotated away for the face of the user to facilitate eating, talking, taking medication, etc.

The respiratory interface device may also further includes a nose bridge pressure adjustment strap having a first end structured to be selectively coupled to one of the headgear and the tube management device and a second end structured to be selectively coupled to the mask, wherein a length of the nose bridge pressure adjustment strap is selectively adjustable to allow for selective adjustment of nose bridge pressure when the connector tubing component is removed from the tube management device such that it is able to hand downwardly from the chin region.

Therefore, it should now be apparent that the invention substantially achieves all the above aspects and advantages. Additional aspects and advantages of the invention will be set forth in the description that follows, and in part will be obvious from the description, or may be learned by practice of the invention. Moreover, the aspects and advantages of the invention may be realized and obtained by means of the instrumentalities and combinations particularly pointed out in the appended claims.

### BRIEF DESCRIPTION OF THE DRAWINGS

FIG. 1 is a schematic illustration of a respiratory interface device according to an embodiment of the invention which illustrates how the respiratory interface device is able to manage and adjust the pressure that is applied to the user's nose;

FIG. 2 is a schematic illustration of a respiratory interface device according to one particular embodiment of the invention;

FIG. 3 is a schematic illustration of a respiratory interface device according to another, alternative particular embodiment of the invention;

FIG. 4 is a schematic illustration of a particular tube management device for use in a respiratory interface device according to a further alternative embodiment of the invention;

FIG. 5 is a schematic illustration of a portion of particular tube management device for use in a respiratory interface device according to a further alternative embodiment of the invention; and

FIG. 6 is a schematic illustration of an alternative respiratory interface device that employs a nose bridge pressure adjustment strap according to a further alternative embodiment of the invention.

### DESCRIPTION OF THE EXEMPLARY EMBODIMENTS

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As employed, herein, the statement that two or more parts or components are “coupled” together shall mean that the parts are joined or operate together either directly or through one or more intermediate parts or components.

As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

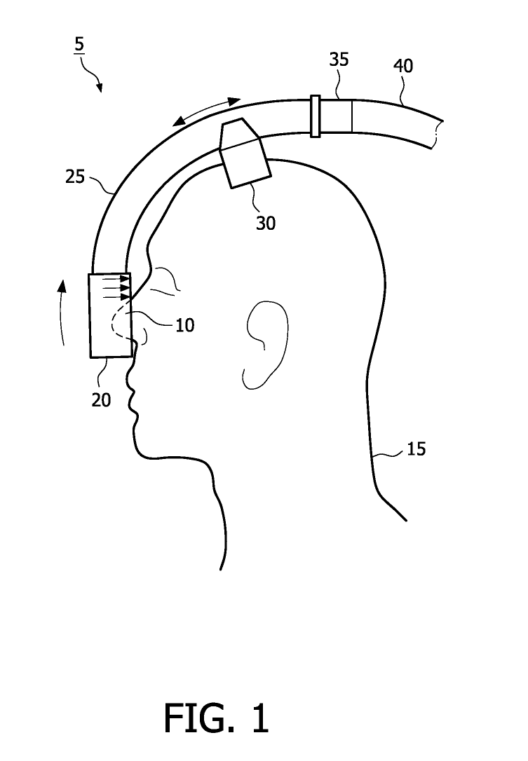

FIG. 1 is a schematic illustration of respiratory interface device 5 according to an embodiment of the invention which illustrates how respiratory interface device 5 is able to manage and adjust the pressure that is applied to the nose 10 of a user 15. As seen in FIG. 1, respiratory interface device 5 includes mask 20 which, when worn by the user 10, applies a force, indicated by the arrows, to the bridge of the nose 10. In the illustrated exemplary embodiment, mask 20 is a nasal mask. It is to be understood, however, that mask 20 can include, without limitation, a nasal/oral mask, nasal pillows, or any other device that provides a suitable gas flow communicating function. Thus, as used herein, the term “mask” shall refer any of such devices.

Respiratory interface device 5 further includes connector tubing 25 coupled to mask 20 at a first end of connector tubing 25. Connector tubing 25 is moveably received within a tube management device 30. Tube management device 30 is structured to be held by headgear (not shown in FIG. 1) forming a part of respiratory interface device 5. As seen in FIG. 1, connector tubing 25 is moveably received within tube management device 30 so as to allow for floating adjustment of connector tubing 25 relative to tube management device 30 in an arced fashion indicated by the arrows of FIG. 1. As connector tubing 25 is moved in this fashion, mask 20, by virtue of being coupled to connector tubing 25, will be caused to rotate relative to nose 10 of user 15 as shown by the arrows in FIG. 1. This rotation will alter/adjust the degree and/or location of the pressure that is applied to the bridge of nose 10 of user 15 by mask 20. Thus, by selectively adjusting the position of connector tubing 25 within tube management device 30, the degree and/or location of the pressure that is applied to the bridge of nose 10 of user 15 by mask 20 can be selectively adjusted as desired. This selective adjustment may result in a better fit (e.g., seal) to the face of user 15, more comfort for user 15, or both.

In addition, connector tubing 25 includes swivel mechanism 35 provided at the second end of connector tubing 25. Swivel mechanism 35 is in turn coupled to supply tube 40 at a first end of supply tube 40. Supply tube 40 is then coupled to a pressure support device (not shown) for providing gas to patient interface device 5. Swivel mechanism 35 allows connector tubing 25 to rotate freely relative to supply tube 40. This is advantageous for a number of reasons, including (i) serving as a torque relief for the mask 20 by reducing the twisting/kinking of the supply tube 40, and (ii) allowing for a fine tuning adjustment for varying nose bridge pressure for the purposes of sealing and comfort as described in greater detail elsewhere herein.

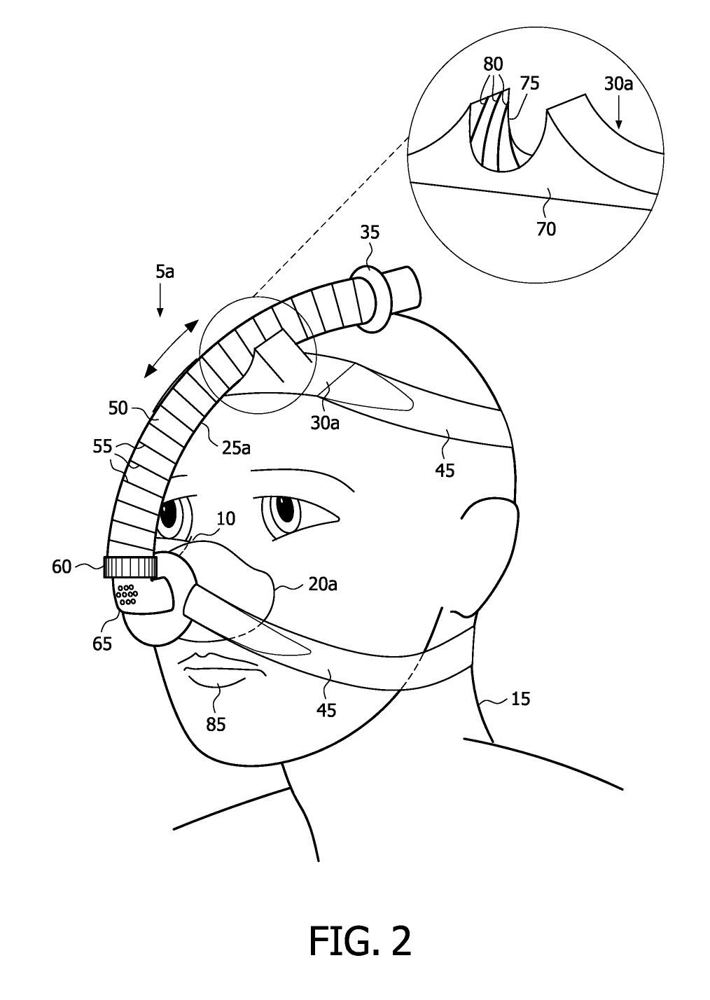

FIG. 2 is a schematic illustration of respiratory interface device 5a according to one particular embodiment of the invention. Respiratory interface device 5a includes nasal mask 20a (other types of masks as described elsewhere herein are also possible) coupled to headgear 45 for attaching respiratory interface device 5a to the head of user 15. Although a particular headgear 45 is shown, it is to be understood that that is not mean tot be limiting, and that other headgear types and styles may also be employed. Respiratory interface device 5a includes connector tubing 25a, which in this embodiment comprises main tubing portion 50 having helical edge 55 extending along the length thereof (the significance of which will be explained below). Connector tubing 25a also includes swivel mechanism 60 provided at the first end thereof. Swivel mechanism 60 is coupled to elbow coupling device 65, which in turn is coupled to mask 20a. Swivel mechanism 60 allows connector tubing 25a to rotate freely relative to mask 20a and elbow coupling device 65. Connector tubing 25a also includes swivel mechanism 35 as described in connection with FIG. 1 to allow connector tubing 25a to rotate freely relative to a supply tube (not shown) coupled thereto.

Respiratory interface device 5a further includes a particular tube management device 30a (shown both attached to the headgear 45 and in a focused view in FIG. 2). The particular tube management device 30a of the embodiment of FIG. 2 includes main body 70 defining central recess 75 for receiving main tubing portion 50 of connector tubing 25a. As seen in the focused view of FIG. 2, tube management device 30a is threaded. In particular, a plurality of threads 80 are provided in central recess 75. Threads 80 are structured to interact and cooperate with helical edge 55 of main tubing portion 50 of connector tubing 25a such that connector tubing 25a can be selectively moved within tube management device 30a by rotating swivel mechanism 60 and thereby rotating main tubing portion 50.

In exemplary embodiment, as shown in FIG. 2, swivel mechanism 60 is provided with raised ridges to facilitate gripping and rotation by providing a tactile element. In addition, to further facilitate gripping and rotation, swivel mechanism 60 can be made of two materials (e.g., by a molding process that includes a double shot), such as a thermoplastic on the inside and a thermoplastic elastomer on the outside. Depending upon the direction in which swivel mechanism 60 is rotated, main tubing portion 50 can be caused to move in either direction within tube management device 30a. As described elsewhere herein, as main tubing portion 50 is moved in this fashion, mask 20a, by virtue of being coupled to connector tubing 25a as just described, will be caused to rotate relative to nose 10 of user 15, and this rotation will alter/adjust the degree and/or location of the pressure that is applied to the bridge of nose 10 of user 15 by mask 20a.

In addition, the threaded nature of tube management device 30a provides for fine adjustment (infinite) of the position of main tubing portion 50. The degree of such fine adjustment (i.e., how much movement will result with each rotation) will be determined by the pitch of threads 80 within central recess 75 and the corresponding spacing helical edge 55 along the length of main tubing portion 50. Furthermore, stop mechanisms may be provided at either end of the main tubing portion 50 to limit movement. Furthermore, such stop mechanisms may be selectively moveable along the length of main tubing portion 50 such that they can be moved into place next to tube management device 30a after the desired position is achieved. In this way, the desired position can be marked so that that specific position can be readily re-attained after connector tubing 25a has been removed as described elsewhere herein.

In addition, connector tubing 25a can be removed from tube management device 30a as desired by applying a force substantially normal to central recess 75. This provides two functions. First, after being so removed, connector tubing 25a can be selectively repositioned within tube management device 30a, thus providing for a gross adjustment of the position of connector tubing 25a and as a result a gross adjustment of the degree and/or location of the pressure that is applied to the bridge of nose 10 of user 15 by mask 20a. Furthermore, when connector tubing 25a is removed from tube management device 30a as just described, mask 20a can be substantially rotated away from the face of user 15 (connector tubing 25a can hang downwardly from the chin region), thereby providing greater access to nose 10 and mouth 85 of user 15 to facilitate eating, talking, taking medication, etc. (as will be appreciated, this feature will be amplified in a case where mask 20a is a nasal/oral mask or a full face mask). Thus, tube management device 30a provides a quick release feature for respiratory interface device 5a to allow for quick and easy removal of connector tubing 25a.

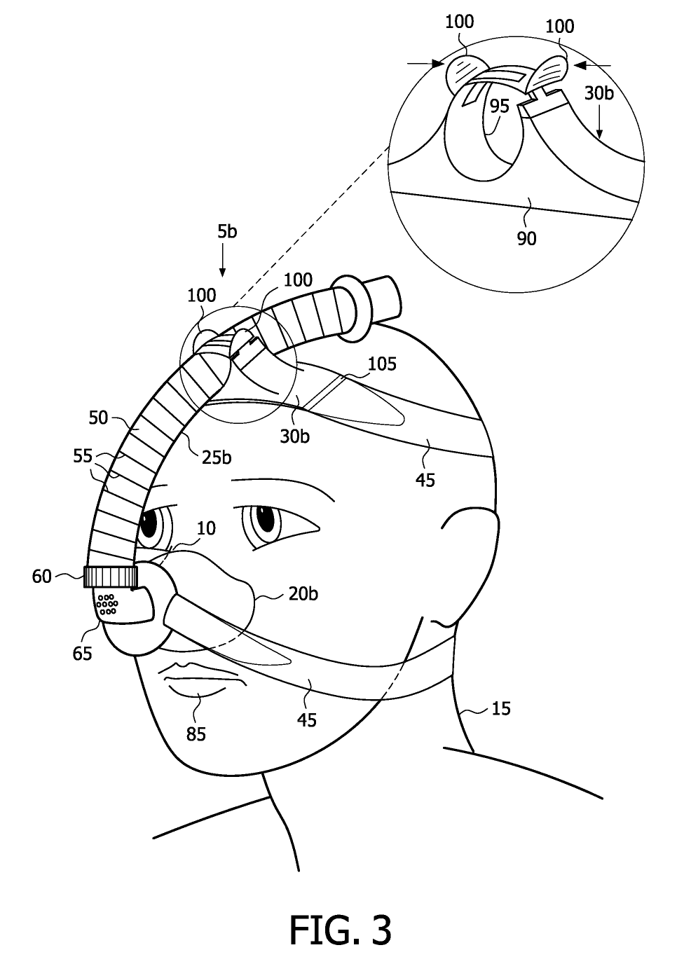

FIG. 3 is a schematic illustration of respiratory interface device 5b according to another, alternative particular embodiment of the invention. Respiratory interface device 5b includes nasal mask 20b (other types of masks as described elsewhere herein are also possible) coupled to headgear 45 for attaching respiratory interface device 5b to the head of user 15. Respiratory interface device 5b includes connector tubing 25a, which in the embodiment shown (which is similar to that shown in FIG. 2) comprises main tubing portion 50 having helical edge 55 extending along the length thereof. It should be understood, however, that the particular main tubing portion 50 shown in FIG. 3 is exemplary only, and that other tubing types, such as, without limitation, simple cylindrical tubing without a helical edge 55, may also be employed.

Connector tubing 25b also includes swivel mechanism 60 provided at the first end thereof. Swivel mechanism 60 is coupled to elbow coupling device 65, which in turn is coupled to mask 20b. Swivel mechanism 60 allows connector tubing 25b to rotate freely relative to mask 20b and elbow coupling device 65. In an alternative embodiment, swivel mechanism 60 may be omitted, in which case main tubing portion 50 would be connected directly to elbow coupling device 65. Connector tubing 25b also includes swivel mechanism 35 as described in connection with FIG. 1 to allow connector tubing 25b to rotate freely relative to a supply tube (not shown) coupled thereto.

Respiratory interface device 5b further includes a particular tube management device 30b (shown both attached to headgear 45 on top of pad 105 and in a focused view in FIG. 3). As described in greater detail below, tube management device 30b employs a friction fit to hold connector tubing 25b. The particular tube management device 30b of the embodiment of FIG. 3 includes main body 90 defining central recess 95 for receiving main tubing portion 50 of connector tubing 25b. As seen in the focused view of FIG. 3, tube management device 30b is not threaded, but instead has a generally uniform surface. In addition, in the exemplary embodiment shown, tube management device 30b includes pressure adjustment tabs 100 which when squeezed in the direction of the arrows causes the force applied to main tubing portion 50 by tube management device 30b to be reduced so that main tubing portion 50 can be freely moved within tube management device 30b. When pressure adjustment tabs 100 are released, main tubing portion 50 will once again be held within tube management device 30b by friction forces. In an exemplary embodiment, when pressure adjustment tabs 100 are in their static condition (i.e., not being squeezed), tube management device 30b applies a friction force such that at least 3 lbs. (or at least 4 lbs.) of pulling force generally along the longitudinal axis of main tubing portion 50 is required to move main tubing portion 50 within tube management device 30b.

As described elsewhere herein, as main tubing portion 50 is moved within tube management device 30b in either fashion just described, mask 20b, by virtue of being coupled to the connector tubing 25b, will be caused to rotate relative to nose 10 of user 15, and this rotation will alter/adjust the degree and/or location of the pressure that is applied to the bridge of nose 10 of user 15 by mask 20b. In the exemplary embodiment, the ability to move main tubing portion 50 within tube management device 30b using only forces of at least 3 lbs. or 4 lbs. will ensure that main tubing portion 50 moves relatively slowly and in a controlled manner, thereby providing a degree of controlled fine adjustment.

In an alternative friction fit embodiment, pressure adjustment tabs 100 may be omitted such that tube management device 30b will have an open top (see, for example, tube management device 30a) while still holding main tubing portion 50 with friction forces, such that at least 3 lbs. (or at least 4 lbs.) of pulling force generally along the longitudinal axis of main tubing portion 50 is required to move main tubing portion 50 within tube management device 30b. In this alternative embodiment, due to the open nature of tube management device 30b, connector tubing 25b can be removed from tube management device 30b by applying a force normal to central recess 95. As was the case with the embodiment of FIG. 2, this provides two functions.

First, after being so removed, connector tubing 25b can be selectively repositioned within tube management device 30b, thus providing for the gross adjustment of the position of connector tubing 25b and as a result a gross adjustment of the degree and/or location of the pressure that is applied to the bridge of nose 10 of user 15 by mask 20b. Furthermore, when connector tubing 25b is removed from tube management device 30b as just described, mask 20b can be substantially rotated away from the face of user 15 (connector tubing 25b can hang downwardly from the chin region), thereby providing greater access to nose 10 and mouth 85 of user 15 to facilitate eating, talking, taking medication, etc. (as will be appreciated, this feature will be amplified in a case where mask 20b is a nasal/oral mask or a full face mask). Thus, tube management device 30b in this alternative embodiment, like tube management device 30a, provides a quick release feature for respiratory interface device 5b to allow for quick and easy removal of connector tubing 25b.

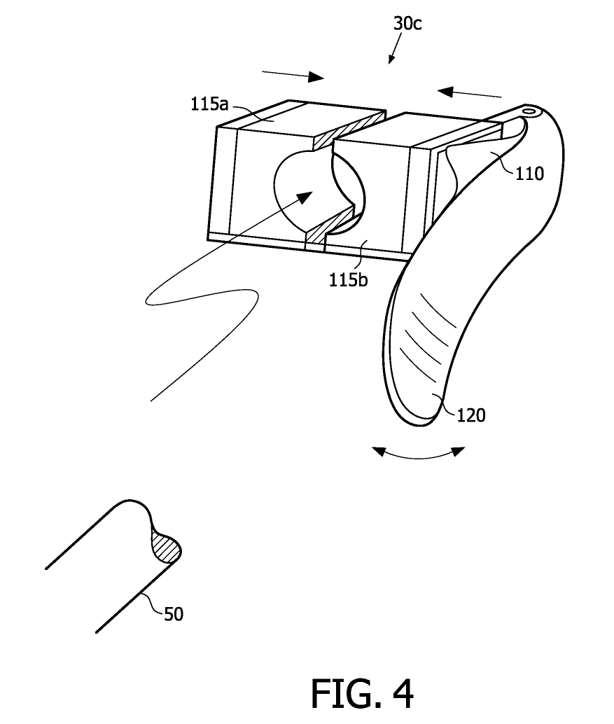

FIG. 4 is a schematic illustration of a particular tube management device 30c according to a further alternative embodiment that may be used in the respiratory interface devices described herein to hold main tubing portion 50 thereof and to allow main tubing portion 50 to be selectively moved therein in order to alter/adjust the degree and/or location of the pressure that is applied to the bridge of nose 10 of user 15 by the associated mask as described elsewhere herein. As seen in FIG. 4, tube management device 30c comprises a cam mechanism that employs cam 110 connected to lever 120 to selectively impart a clamping force onto main tubing portion 50 when it is inserted between two moveable tube holders 115a and 115b. When lever 120 is rotated away from tube holders 115a and 115b, the clamping force is reduce/removed, and as a result, the position of main tubing portion 50 can be finely adjusted within tube management device 30c. When main tubing portion 50 is positioned as desired, lever 120 can be rotated toward tube holders 115a and 115b to cause the clamping force to be applied to main tubing portion 50 to securely hold it in its current position.

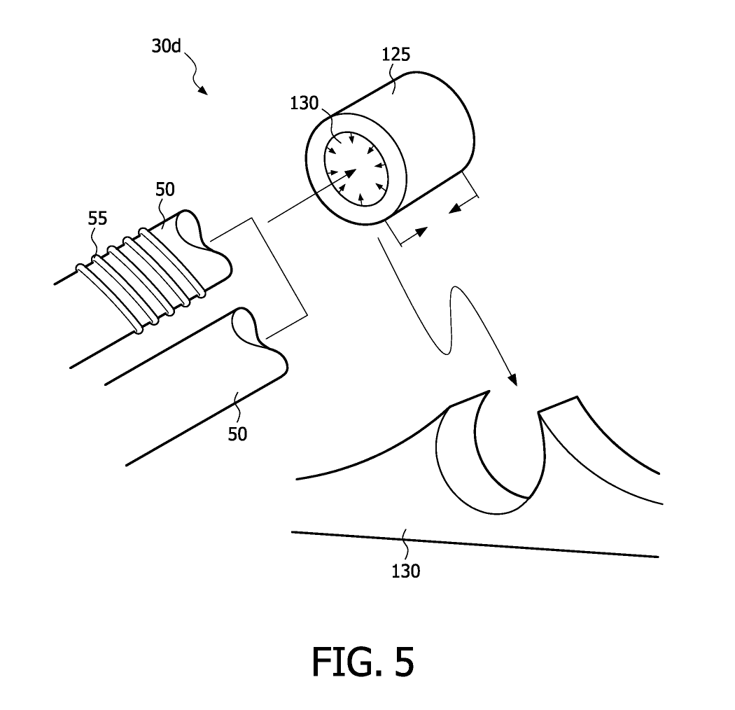

FIG. 5 is a schematic illustration of a portion of particular tube management device 30d according to a further alternative embodiment that may be used in the respiratory interface devices described herein to hold main tubing portion 50 thereof (two such alternative main tubing portions 50 are shown in FIG. 5) and to allow main tubing portion 50 to be selectively moved/positioned relative to headgear 45 in order to alter/adjust the degree and/or location of the pressure that is applied to the bridge of nose 10 of user 15 by the associated mask as described elsewhere herein. Tube management device 30d includes generally cylindrical tube attachment piece 125 that can be selectively positioned along the length of main tubing portion 50. Tube attachment piece 125 is made of a foam material, a low durometer rubber, or another suitable compressible material.

As seen in FIG. 5, tube attachment piece 125 includes internal diameter 130 in which main tubing portion 50 may be inserted. In addition, tube attachment piece 125 is structured such that when a compression force is applied as indicated by the arrows shown adjacent to the tube attachment piece 125 in FIG. 5, internal diameter 130 of tube attachment piece 125 will be reduced as indicated by the arrows within tube attachment piece 125. The compression force may be applied in a number of different ways, such as by the fingers of the user, or as a result of the rotation of a screw mechanism (not shown), which in turn causes the compression force to be imparted on tube attachment piece 125. This enables tube attachment piece 125 to be secured to a particular location along the length of main tubing portion 50. Tube attachment piece 125, once so secured in place, can then be inserted into holding mechanism 130, such as a friction fit holding mechanism as described elsewhere herein, provided on headgear 45. As will be appreciated, this allows main tubing portion 50 to be selectively moved/positioned relative to headgear 45 to selectively adjust the nose bridge pressure. Furthermore, as will also be appreciated, tube management device 30d as just described provides a quick release feature for the respiratory interface device in which it is employed as described elsewhere herein.

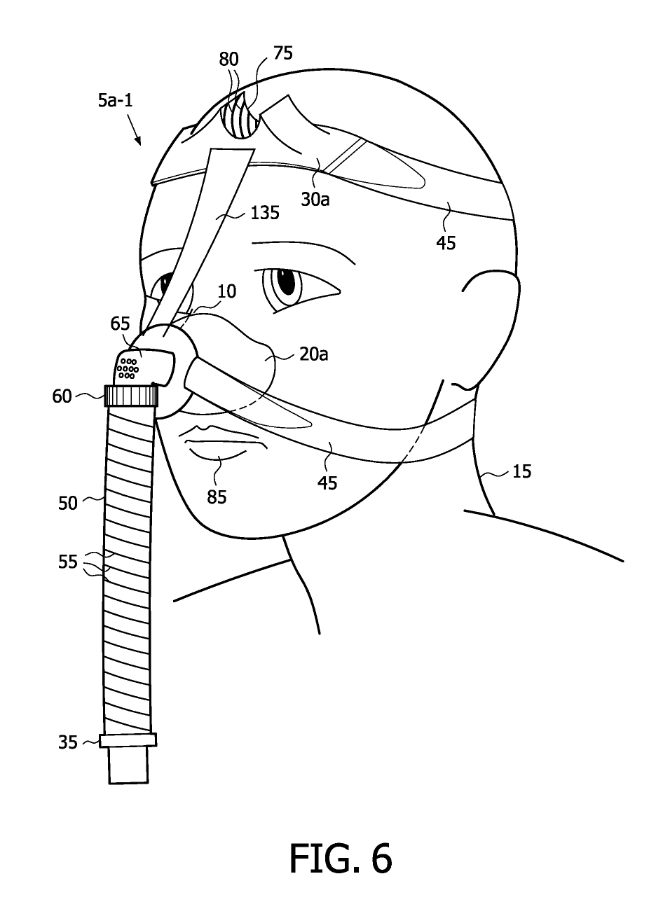

FIG. 6 is a schematic illustration of an alternative version of respiratory interface device 5a shown in FIG. 2, identified as 5a-1. Thus, as seen in FIG. 6, many of the same parts and components are included. However, respiratory interface device 5a-1 is provided for users who, at times, prefer not to have connector tubing 25a extend up over their head. Thus, respiratory interface device 5a-1 is provided with a further accessory in the form of nose bridge pressure adjustment strap 135. As seen in FIG. 6, nose bridge pressure adjustment strap 135 extends between a connector (also used for attaching headgear 45) of mask 20a and tube management device 30a (alternatively, nose bridge pressure adjustment strap 135 may be connected directly to headgear 45). The length of nose bridge pressure adjustment strap 135 may be selectively adjusted using, in the exemplary embodiment, Velcro® adjustment mechanisms (other suitable adjustment mechanism may also be used).

As will be appreciated, when the length of nose bridge pressure adjustment strap 135 is so adjusted, mask 20a will be caused to rotate/move relative to nose 10 of user 15, and this rotation/movement will alter/adjust the degree and/or location of the pressure that is applied to the bridge of nose 10 of user 15 by mask 20a. In addition, as shown in FIG. 6, when nose bridge pressure adjustment strap 135 is employed, elbow coupling device 65 may be rotated into a position wherein connector tubing 25a is allowed to extend downwardly from the face of user 15 rather than up over the head of user 15. While nose bridge pressure adjustment strap 135 has been described in connection with the particular embodiment that employs the threaded tube management device 30a, it should be understood that this is meant to be exemplary only and that nose bridge pressure adjustment strap 135 may also be used with the other embodiments (e.g., FIGS. 3-5) described elsewhere herein.

While preferred embodiments of the invention have been described and illustrated above, it should be understood that these are exemplary of the invention and are not to be considered as limiting. Additions, deletions, substitutions, and other modifications can be made without departing from the spirit or scope of the present invention. Accordingly, the invention is not to be considered as limited by the foregoing description but is only limited by the scope of the appended claims.

What is claimed is:

1. A respiratory interface device, comprising:
a mask structured to engage a bridge of a nose of a user;
a connector tubing component, a first end of the connector tubing component being coupled to the mask, the connector tubing component having a main tubing portion and a swivel mechanism provided at a second end of the connector tubing component for coupling the connector tubing component to a gas supply tube in a manner that permits the connector tubing component and the gas supply tube to rotate relative to one another;
a headgear; and
a tube management device coupled to the headgear, wherein the main tubing portion of the connector tubing component is structured to be moveably received within and held by the tube management device, and wherein a pressure applied to the bridge of the nose of the user by the mask may be selectively adjusted by adjusting a position of the main tubing portion within the tube management device.

2. The respiratory interface device according to claim 1, further comprising a second swivel mechanism provided at the first end of the connector tubing component for coupling the connector tubing component to the mask in a manner that permits the connector tubing component and the mask to rotate relative to one another.

3. The respiratory interface device according to claim 2, wherein the tube management device includes a recess having a plurality of threads for receiving the main tubing portion, wherein the main tubing portion has an edge extending along at least a portion of a length thereof for cooperating with the plurality of threads, and wherein the main tubing portion is structured to move within the tube management device in response to rotation of the second swivel mechanism as a result of the cooperation between the edge and the plurality of threads.

4. The respiratory interface device according to claim 3, wherein the edge is a helical edge.

5. The respiratory interface device according to claim 3, wherein the connector tubing component is removable from the tube management device in response to a pulling force being applied to the connector tubing component in a direction that is substantially normal to the tube management device.

6. The respiratory interface device according to claim 1, wherein the main tubing portion is held within the tube management device by a friction force, and wherein as a result of the friction force the main tubing portion is structured to move within the tube management device in a direction substantially along a longitudinal axis of the main tubing portion only in response to a pulling force of at least 3 lbs.

7. The respiratory interface device according to claim 6, wherein the main tubing portion is structured to move within the tube management device in a direction substantially along the longitudinal axis of the main tubing portion only in response to a pulling force of at least 4 lbs.

8. The respiratory interface device according to claim 6, wherein the tube management device includes a pair of pressure adjustment tabs which when squeezed toward one another reduce the friction force applied to the main tubing portion such that the main tubing portion is able to freely move within the tube management device.

9. The respiratory interface device according to claim 6, further comprising a second swivel mechanism provided at the first end of the connector tubing component for coupling the connector tubing component to the mask in a manner that permits the connector tubing component and the mask to rotate relative to one another.

10. The respiratory interface device according to claim 6, wherein the connector tubing component is removable from the tube management device in response to a pulling force being applied to the connector tubing component in a direction that is substantially normal to the tube management device.

11. The respiratory interface device according to claim 1, wherein the tube management device comprises a cam mechanism that employs a cam connected to a lever to selectively impart a clamping force onto the main tubing portion.

12. The respiratory interface device according to claim 11, further comprising a second swivel mechanism provided at the first end of the connector tubing component for coupling the connector tubing component to the mask in a manner that permits the connector tubing component and the mask to rotate relative to one another.

13. The respiratory interface device according to claim 1, further comprising a selectively compressible tube attachment piece selectively attachable to the main tubing portion at a desired position along a length thereof, wherein the main tubing portion is coupled to the tube management device through the selectively compressible tube attachment piece.

14. The respiratory interface device according to claim 13, further comprising a second swivel mechanism provided at the first end of the connector tubing component for coupling the connector tubing component to the mask in a manner that permits the connector tubing component and the mask to rotate relative to one another.

15. The respiratory interface device according to claim 13, wherein the connector tubing component is removable from the tube management device in response to a pulling force being applied to the connector tubing component in a direction that is substantially normal to the tube management device.

16. The respiratory interface device according to claim 1, wherein the connector tubing component is removable from the tube management device in response to a pulling force being applied to the connector tubing component in a direction that is substantially normal to the tube management device, and wherein respiratory interface device further includes a nose bridge pressure adjustment strap having a first end structured to be selectively coupled to one of the headgear and the tube management device and a second end structured to be selectively coupled to the mask, wherein a length of the nose bridge pressure adjustment strap is selectively adjustable.
